(19) United States
(12) Patent Application Publication
(10) Pub. No.: US 2023/0284927 A1
(43) Pub. Date: Sep. 14, 2023

(54) INJECTIONLESS CONDUCTANCE METHOD FOR VASCULAR SIZING

(71) Applicant: Ghassan S. Kassab, La Jolla, CA (US)

(72) Inventor: Ghassan S. Kassab, La Jolla, CA (US)

(21) Appl. No.: 18/084,900

(22) Filed: Dec. 20, 2022

Related U.S. Application Data
(63) Continuation of application No. 16/368,768, filed on Mar. 28, 2019, now abandoned.
Application No. 16/368,768 is a continuation-in-part of application No. 16/323,136, filed on Feb. 4, 2019, now abandoned.
(60) Provisional application No. 62/649,558, filed on Mar. 28, 2018.
(60) Provisional application No. 62/371,045, filed on Aug. 4, 2016.

(51) Int. Cl. A61B 5/0538; A61B 5/0215; A61B 5/107; A61B 5/00; A61B 90/00

(52) U.S. Cl. CPC A61B 5/0538; A61B 5/02158; A61B 5/1076; A61B 5/6851; A61B 5/6853; A61B 90/06; A61B 5/1079; A61B 5/02007

(57) ABSTRACT

A method of determining the diameter of a luminal organ. The method utilizing the introduction of two different frequencies into the lumen of the organ via an impedance catheter/guidewire to obtain conductance measurements and not requiring a separate fluid injection. The method utilizes the presence of a fluid native to the luminal organ.

### RELATED APPLICATIONS

The present application is related to, claims the priority benefit of, and is a U.S. continuation patent application of, U.S. patent application Ser. No. 16/368,768, filed Mar. 28, 2019, which a) is related to, and claims the priority benefit of, U.S. Provisional Patent Application Ser. No. 62/649,558, filed Mar. 28, 2018, and b) is related to, claims the priority benefit of, and is a U.S. continuation-in-part patent application of, U.S. patent application Ser. No. 16/323,136, filed Feb. 4, 2019, which is related to, claims the priority benefit of, and is the U.S. national stage § 371 application of, International Patent Application Serial No. PCT/US2017/045581, filed Aug. 4, 2017, which is related to, and claims the priority benefit of, U.S. Provisional Patent Application Ser. No. 62/371,045, filed Aug. 4, 2016. The contents of each of the aforementioned applications are hereby incorporated by reference in their entirety.

### BACKGROUND

Lumen vessel sizing is important for optimization of interventional outcomes for treatment of vascular disease. The clinical significance of accurate sizing of an artery for percutaneous treatment has been well established by numerous randomized clinical trials. Under-sizing causes an increase in restenosis rates and oversizing may cause dissection, perforation, or acute vessel closure. Devices and methods configured to obtain accurate sizing are most useful in the medical arts.

The value of optimal sizing during percutaneous transluminal angioplasty (PTA) has been validated in numerous studies using intravascular ultrasound (IVUS). For PTA, IVUS improves procedural results due to optimal balloon sizing leading to significant improvement in luminal dimensions. Despite the utility of IVUS, it is not used routinely because of the added time, complexity, subjective interpretation of images, cost, and required training associated with its usage.

Angiography (visual estimation or “eye balling” and quantitative angiography (QA)) is used more routinely but uses a 2D slice projection of a 3D vessel and relies upon edge detection (which assumes a circular vessel). Hence, QA lacks accuracy for sizing because of spatial resolution and irregularity of vessel geometry (i.e., non-circular diseased vessels). Incorrect sizing from visual estimation and QA has been shown to lead to suboptimal therapy delivery and diminished patient outcomes.

Pre-clinical and clinical studies have been performed to validate the functionality of the 0.035″ LumenRECON (LR) guidewire (used in various studies referenced herein) as a standard workhorse guidewire for vessel navigation and as an accurate diagnostic tool for luminal sizing in comparison with other imaging modalities (e.g., QA, IVUS and duplex ultrasound). The lumen sizing is performed with two-bolus injections of saline solutions with different salinities (e.g. normal and half normal).

The saline injections add time to the procedure and have some limitations and hence should ideally be eliminated. For example, the need for saline injections may be more troublesome during a standard pullback procedure to determine continuous real-time quantitative measurement of lumen cross sectional area (CSA) in normal and diseased vessels. Furthermore, although normal saline is typically available for flushes from a saline bag on the manifold, half normal saline needs to be poured into a bowl as it is not used in standard procedures. Finally, the assumption of the injection method is that the saline solution will transiently fully displace the blood during the injection. This requires good engagement of the introducing catheter to allow a brisk flush (similar to contrast injection for an angiogram).

An additional underlying assumption is that the parallel conductance, Gₚ, is constant over the injections of two different saline solutions. This assumption may be challenged when Gₚ becomes excessively high (e.g., when over 90% of current is lost through the vessel wall and surrounding tissue).

Although there are several advantages of the electrical conductance technology given the ease of use in comparison with IVUS (real-time measurements, no need for interpretation, etc.) in a recent first in man, the major feedback from the clinicians was to eliminate the need for the half normal saline injection and ideally both (half normal and normal saline). Thus there is a need for a simpler and more efficient noninvasive methodology of measuring luminal parameters.

### BRIEF SUMMARY

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

A methodology was developed to eliminate the need for saline solution injections and to use multiple frequencies to calculate the vessel lumen cross-sectional area. Although similar techniques have been attempted to estimate the left ventricular volume, they have been based on empirical parameters (as compared to the present approach which is entirely physics-based) or have involved the need for parameters that are invasively measured and hence not clinically translational.

The present disclosure includes disclosure of a method, comprising the steps of introducing at least part of an impedance device into a luminal organ at a first location so that a detector of the device is positioned within the luminal organ; introducing a first frequency through the detector of the device and obtaining a first conductance measurement using the detector in connection with the first frequency; introducing a second frequency through the detector of the device and obtaining a second conductance measurement using the detector in connection with the second frequency; and determining a diameter or cross-sectional area at the first location within the luminal organ using the first conductance measurement, the second conductance measurement, the conductivity of fluid within the luminal organ, and a known distance between detection elements of the detector, and without injecting a fluid into the luminal organ in order to obtain the first conductance measurement and the second conductance measurement.

The present disclosure includes disclosure of a method, further comprising the step of generating a size profile of the luminal organ using the determined diameter or cross-sectional area at the first location and at least one additional diameter or cross-sectional area obtained by performing the steps of the method at a second location within the luminal organ.

The present disclosure includes disclosure of a method, wherein the conductivity of fluid within the luminal organ is determined by operating the detector of the device within a catheter positioned within the luminal organ by obtaining a conductance measurement within the catheter having a known diameter.

The present disclosure includes disclosure of a method, wherein the step of introducing at least part of the impedance device is performed to position the at least part of the device into the luminal organ wherein the detector comprises the two detection electrodes positioned in between two excitation electrodes, wherein the known distance between each of the electrodes is 5 mm.

The present disclosure includes disclosure of a method, wherein the steps of introducing the first frequency and introducing the second frequency are performed by operating a frequency generator in communication with the device, the frequency generator selected from the group consisting of an arbitrary waveform generator and multiple signal generators.

The present disclosure includes disclosure of a method, wherein the determining step is further performed to determine a parallel tissue conductance.

The present disclosure includes disclosure of a method, wherein the step of introducing at least part of the impedance device is performed to position the at least part of the device into the luminal organ wherein the detector comprises the two detection electrodes positioned in between two excitation electrodes, wherein the known distance between each of the electrodes is 2 mm.

The present disclosure includes disclosure of a method, wherein the first frequency and the second frequency are the only frequencies introduced.

The present disclosure includes disclosure of a method, further comprising the steps of moving the at least part of the impedance device within the luminal organ to a second location; introducing one of the first frequency or the second frequency through the detector of the device and obtaining a third conductance measurement using the detector in connection with the one of the first frequency or the second frequency; introducing the other of the first frequency or the second frequency through the detector of the device and obtaining a fourth conductance measurement using the detector in connection with the other of the first frequency or the second frequency; determining a second diameter or cross-sectional area at the second location within the luminal organ using the third conductance measurement, the fourth conductance measurement, the conductivity of fluid within the luminal organ, and the known distance between detection elements of the detector, and without injecting a fluid into the luminal organ in order to obtain the third conductance measurement and the fourth conductance measurement; and generating a size profile of the luminal organ using the determined diameter or cross-sectional area at the first location and at the determined second diameter or cross-sectional area at the second location.

The present disclosure includes disclosure of a method, comprising the steps of introducing at least part of an impedance device into a luminal organ so that a detector of the device is positioned within the luminal organ; operating the detector of the device within a catheter having a known diameter to obtain a first conductance measurement, wherein the catheter is positioned within the luminal organ and filled with a fluid native to the luminal organ; operating the detector of the device to obtain a second conductance measurement, wherein the detector is positioned within the luminal organ and out of the catheter; determining an initial estimate of the diameter or cross-sectional area of the luminal organ; and determining an actual diameter or cross-sectional area of the luminal organ based on the first conductance measurement, the second conductance measurement, and the initial estimate of the diameter or cross-sectional area of the luminal organ.

The present disclosure includes disclosure of a method, wherein the steps of operating the detector of the device comprises introducing a first frequency through the detector of the device and introducing a second frequency through the detector of the device.

The present disclosure includes disclosure of a method, wherein the first frequency and the second frequency are the only frequencies introduced.

The present disclosure includes disclosure of a method, performed without injecting any fluid into the luminal organ.

The present disclosure includes disclosure of a method, further comprising the step of calculating at least one parallel conductance measurement based on the initial estimate of the diameter or cross-sectional area of the luminal organ.

The present disclosure includes disclosure of a method, further comprising the step of calculating a second parallel conductance measurement based on the initial estimate of the diameter or cross-sectional area of the luminal organ.

The present disclosure includes disclosure of a method, comprising the steps of operating an impedance device to introduce a signal through the detection device into a luminal organ, the signal comprising a first signal having a first frequency, a second signal having a second frequency, and obtaining output conductance data in connection with each of the two signals using an impedance detector of the impedance device; and determining a diameter or cross-sectional area of the luminal organ based upon the output conductance data in connection with each of the two signals and a conductivity of blood within the luminal organ; wherein the method is performed without injecting any fluid into the luminal organ; and wherein the signals are comprised of only the first signal and the second signal.

The present disclosure includes disclosure of a method, wherein the step of operating the impedance device to introduce a signal is performed repeatedly while the device is in motion.

The present disclosure includes disclosure of a method, wherein the step of operating the impedance device is performed to operate the impedance detector that comprises the two detection electrodes positioned in between two excitation electrodes, wherein the known distance between each of the electrodes is selected from the group consisting of 2 mm and 5 mm.

The present disclosure includes disclosure of a method, further comprising the steps of moving the impedance device within the luminal organ to a second location; operating an impedance device to introduce the signal through the detection device into a luminal organ, the signal comprising the first signal having the first frequency and the second signal having the second frequency, and obtaining additional output conductance data in connection with each of the two signals using the impedance detector of the impedance device; and determining a second diameter or cross-sectional area of the luminal organ based upon the additional output conductance data in connection with each of the two signals and the conductivity of blood within the luminal organ.

The present disclosure includes disclosure of a method, wherein the step of determining the diameter or cross-sectional area of the luminal organ is performed based upon an initial estimate of the luminal organ diameter or cross-sectional area.

The present disclosure includes disclosure of an injection-less method to determine the lumen diameter, using multiple frequencies, specifically using only two frequencies, that eliminates the need for saline injections and can utilize the same electrical conductance devices developed for the two-injection method. A mathematical electrical model was devised to estimate the lumen area and diameter of the arteries. In vitro experiments were used to validate the method for various lumen diameters with both 5-5-5 (for use in peripheral vessels) and 2-2-2 (for use in coronary vessels) spacing (where the numbers are referring to the spacings, in millimeters, between each electrode or sensor, such as from the distal excitation electrode to the adjacent detection electrode to the next adjacent detection electrode to the proximal excitation electrode, for example) conductance/sizing guidewires. The majority of the experiment's 11 vessel's data fall within one standard deviation and all the data fall within two standard deviations. The results indicate that the two-frequency model can reasonably predict the lumen diameter in in vitro tests set-up and this approach can translate to in vivo which would reduce the time of the measurement and enable pull-back to reconstruct the dimensional profile of the vessel lumen.

In an embodiment for a method of determining the diameter of a luminal organ, the method comprises the steps of introducing at least part of an impedance device into a luminal organ at a first location so that a detector of the device is positioned within the luminal organ; introducing a first frequency through the detector of the device and obtaining a first conductance measurement using the detector in connection with the first frequency; introducing a second frequency through the detector of the device and obtaining a second conductance measurement using the detector in connection with the second frequency; and determining a diameter at the first location within the luminal organ using the first conductance measurement, the second conductance measurement, the conductivity of fluid within the luminal organ, and a known distance between detection elements of the detector.

In another embodiment the method further comprises the step of generating a size profile of the luminal organ using the determined cross-sectional area at the first location and at least one additional cross-sectional area obtained by performing the steps of the method at a second location within the luminal organ.

In another embodiment the conductivity of fluid within the luminal organ is determined by operating the detector of the device within a catheter positioned within the luminal organ by obtaining a conductance measurement within the catheter having a known diameter.

In another embodiment the method further comprises the step of introducing at least part of the impedance device is performed to position the at least part of the device into the luminal organ wherein the detector comprises the two detection electrodes positioned in between two excitation electrodes, wherein the known distance between each of the electrodes is 5 mm.

In another embodiment of the method the steps of introducing the first frequency and introducing the second frequency are performed by operating a frequency generator in communication with the device, the frequency generator selected from the group consisting of an arbitrary waveform generator and multiple signal generators.

In another embodiment the determining step is performed to determine a parallel tissue conductance.

In another embodiment the step of introducing at least part of the impedance device is performed to position the at least part of the device into the luminal organ wherein the detector comprises the two detection electrodes positioned in between two excitation electrodes, wherein the known distance between each of the electrodes is 2 mm.

In another embodiment the first frequency and the second frequency are the only frequencies introduced.

In another embodiment the method is performed without injecting any fluid into the mammalian luminal organ.

In an embodiment for a method of determining the diameter of a luminal organ, the method comprises the steps of introducing at least part of an impedance device into a luminal organ so that a detector of the device is positioned within the luminal organ; operating the detector of the device within a catheter having a known diameter to obtain a first conductance measurement, wherein the catheter is positioned within the luminal organ and filled with a fluid native to the luminal organ; operating the detector of the device to obtain a second conductance measurement, wherein the detector is positioned within the luminal organ and out of the catheter; determining an initial estimate of the diameter of the luminal organ; and determining a diameter of the luminal organ based on the first conductance measurement, the second conductance measurement and an initial estimate of the diameter of the luminal organ.

In another embodiment the steps of operating the detector of the device comprises introducing a first frequency through the detector of the device and introducing a second frequency through the detector of the device.

In another embodiment the method is performed to calculate at least one parallel conductance measurement based on the initial estimate of the diameter of the luminal organ.

In an embodiment for a method of determining the diameter of a luminal organ, the method comprises the steps of: operating an impedance device to introduce a signal through the detection device into a luminal organ, the signal comprising a first signal having a first frequency, a second signal having a second frequency, and obtaining output conductance data in connection with each of the two signals using an impedance detector of the impedance device; and determining a diameter of the luminal organ based upon the output conductance data in connection with each of the two signals and a conductivity of blood within the luminal organ.

In another embodiment the step of operating the impedance device to introduce a signal is performed repeatedly while the device is in motion.

In another embodiment the signal is comprised of only the first signal and the second signal.

In another embodiment the method is performed without injecting any fluid into the luminal organ, and the stimulating signal is comprised of only the first signal and the second signal.

In another embodiment the step of determining a diameter of the luminal organ is performed based upon an initial estimate of the luminal organ diameter.

### BRIEF DESCRIPTION OF THE DRAWINGS

The disclosed embodiments and other features, advantages, and disclosures contained herein, and the matter of attaining them, will become apparent and the present disclosure will be better understood by reference to the following description of various exemplary embodiments of the present disclosure taken in conjunction with the accompanying drawings, wherein:

FIG. 1 shows steps of an exemplary method in flowchart form, according to an exemplary embodiment of the present disclosure.

FIG. 2A shows an exemplary system for obtaining a parallel tissue conductance within a luminal organ according to an embodiment of the present disclosure;

FIG. 2B shows an exemplary detection device of an exemplary system for obtaining a parallel tissue conductance within a luminal organ having impedance measuring electrodes supported in front of a stenting balloon thereon, according to an embodiment of the present disclosure;

FIG. 2C shows an exemplary detection device of an exemplary system for obtaining a parallel tissue conductance within a luminal organ having impedance measuring electrodes within and in front of a balloon thereon, according to an embodiment of the present disclosure;

FIG. 2D shows an exemplary detection device of an exemplary system for obtaining a parallel tissue conductance within a luminal organ having an ultrasound transducer within and in front of a balloon thereon, according to an embodiment of the present disclosure;

FIG. 2E shows an exemplary detection device of an exemplary system for obtaining a parallel tissue conductance within a luminal organ without a stenting balloon, according to an embodiment of the present disclosure;

FIG. 2F shows an exemplary detection device of an exemplary system for obtaining a parallel tissue conductance within a luminal organ having wire and impedance electrodes, according to an embodiment of the present disclosure;

FIG. 2G shows an exemplary detection device of an exemplary system for obtaining a parallel tissue conductance within a luminal organ having multiple detection electrodes, according to an embodiment of the present disclosure;

FIGS. 2H, 2I, and 2J show at least a portion of exemplary systems for obtaining a parallel tissue conductance within a luminal organ according to embodiments of the present disclosure;

FIG. 3 shows steps of an exemplary method for obtaining a diameter and parallel tissue conductance within a luminal organ using a method according to an embodiment of the present disclosure;

FIG. 4 shows steps of another exemplary method for obtaining a diameter and parallel tissue conductance within a luminal organ according to an embodiment of the present disclosure;

FIG. 5 shows a saline solution conductivity correction factor (BCCF) as function of lumen diameter for two different guidewire diameters, wherein BCCF is defined as the ratio of real to ideal conductivities of the blood, according to an exemplary embodiment of the present disclosure;

FIG. 6 shows a vessel wall tissue conductivity correction factor (TCCF) as function of lumen diameter (TR=0.4), wherein the TCCF is defined as the ratio of real to ideal conductivities of the tissue, according to an exemplary embodiment of the present disclosure;

FIG. 7 shows the ratio of parallel conductance to the conductance inside the lumen for an infinite tissue thickness as function of lumen diameter, wherein the difference in the plots are due to the difference of conductivity inside the lumen, according to an exemplary embodiment of the present disclosure;

FIG. 8 shows a predicted lumen diameter versus optically measured for the 11 in vitro tests with the line of equality, according to an exemplary embodiment of the present disclosure;

FIG. 9 shows a Bland-Altman chart for the 11 in vitro tests, according to an exemplary embodiment of the present disclosure;

FIG. 10 shows a comparison of measured parallel conductance at 10 and 20 KHz, with model prediction as function of bath thickness, according to an exemplary embodiment of the present disclosure;

FIG. 11 shows a ratio of parallel conductance to the total conductance for various lumen diameters for 5-5-5 guidewire based on model prediction, noting that there is 0.45% saline solution in the lumen and 0.1% saline solution in the tissue which closely represent the in vivo condition, according to an exemplary embodiment of the present disclosure;

FIG. 12 shows a ratio of parallel conductance to the total conductance for various lumen diameters for 2-2-2 guidewire based on model predication, noting that there is 0.45% saline solution in the lumen and 0.1% saline solution in the tissue which closely represent the in vivo condition, according to an exemplary embodiment of the present disclosure; and

FIG. 13 shows a ratio of parallel conductance to total conductance as function of vessel diameter for 25 patients with two injections method using 5-5-5 guidewire, with the points shown by cross marks being the values predicted by the model, according to an exemplary embodiment of the present disclosure; and

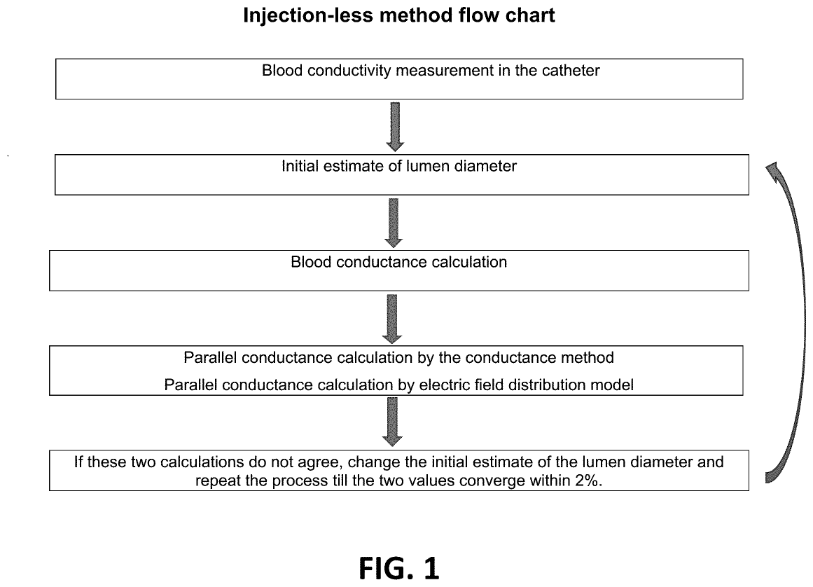

As such, an overview of the features, functions and/or configurations of the components depicted in the various figures will now be presented. It should be appreciated that not all of the features of the components of the figures are necessarily described and some of these non-discussed features (as well as discussed features) are inherent from the figures themselves. Other non-discussed features may be inherent in component geometry and/or configuration. Furthermore, wherever feasible and convenient, like reference numerals are used in the figures and the description to refer to the same or like parts or steps. The figures are in a simplified form and not to precise scale.

### DETAILED DESCRIPTION

The present disclosure details the development a two-frequency method that eliminates the need for saline solution injections. The basic premise is to vary the electrical admittance through various energy (i.e., frequency) rather than salinity. This approach was validated in phantoms and in invitrovessels. The same conductance/sizing guidewire that has been developed for the two-injection method (5-5-5 spacing for peripheral and 2-2-2 guidewire for coronary vessels) was utilized as well. This studies and associated disclosure referenced herein provide for a method to predict the vessel lumen diameter in real-time as well as to allow pullback profiles of vessel lumen with unprecedented accuracy to guide therapy delivery for treatment of vascular disease.

### EXEMPLARY DEVICE

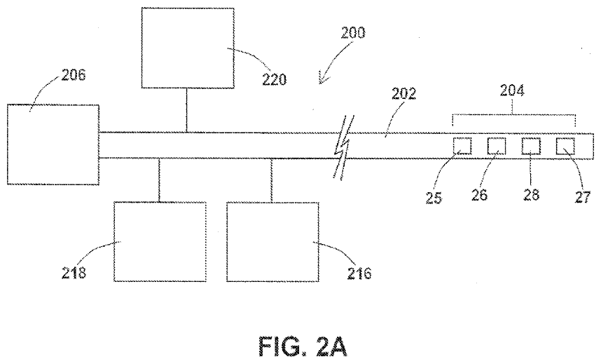

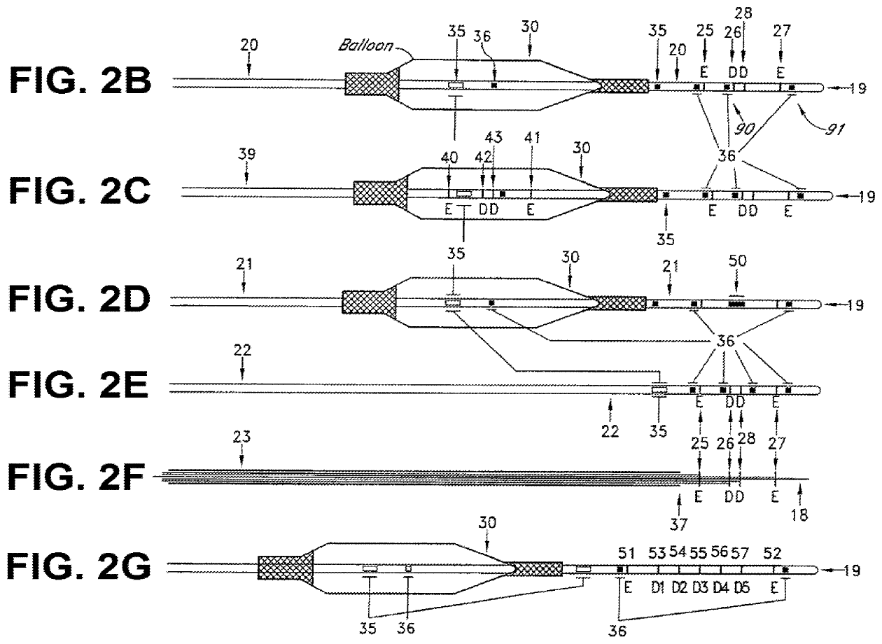

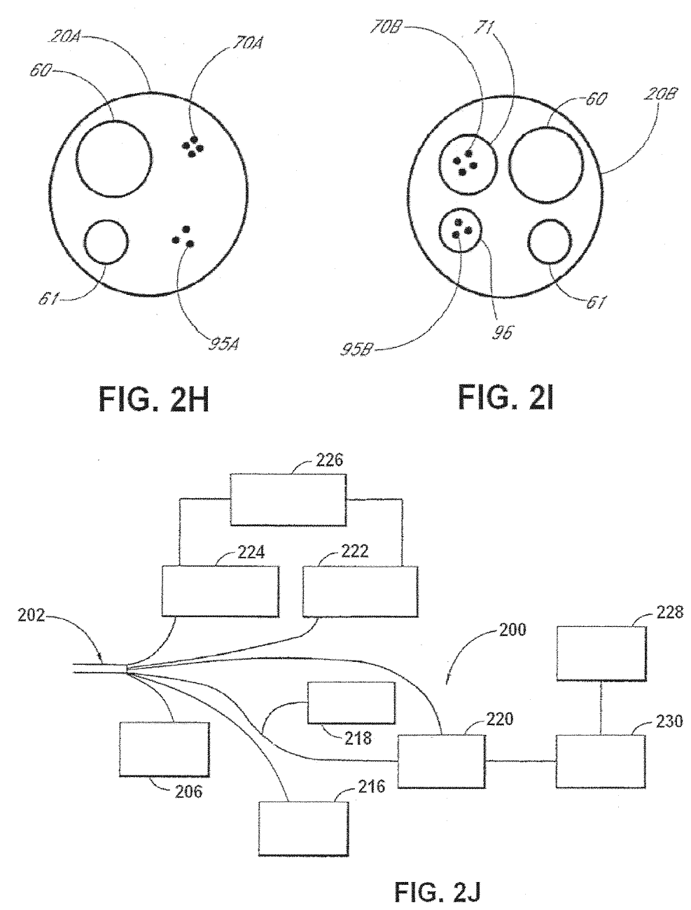

An exemplary system for obtaining a parallel tissue conductance within a luminal organ of the present disclosure is shown in FIG. 2A. As shown in FIG. 2A, an exemplary embodiment of a system 200 of the present disclosure comprises a detection device 202 having a detector 204, and a frequency generator 206 coupled to detection device 202. Frequency generator 206, in at least one embodiment, is capable of generating signals having at least two distinct frequencies through detection device 202. An exemplary frequency generator 206 may include, but is not limited to, an arbitrary waveform generator or two signal generators. In at least one embodiment of an arbitrary waveform generator, the output conductance can be filtered at the appropriate frequency to derive the desired conductance for each frequency. In at least one embodiment of system 200 of the present disclosure, detector 204 comprises detection electrodes 26, 28 positioned in between excitation electrodes 25, 27, wherein excitation electrodes 25, 27 are capable of producing an electrical field.

The spacing between the electrodes is preferably equidistant and preferably either 5 mm or 2 mm between electrodes.

The frequency generator 206 is configured to generate two signals between 0.1-100 kHz. Preferably the signals generated are between 1-100 kHz. In another embodiment, the signals are between 10-80 kHz. In another embodiment, two signals are generated at 10 kHz and 20 kHz. In another embodiment, the frequency generator can generated any combination of two frequencies.

In an exemplary embodiment of system 200, system 200 further comprises a deconvolution device 216, whereby deconvolution device 216 is capable of filtering an output conductance to obtain a first conductance value and a second conductance value from the output conductance, and/or whereby deconvolution device 216 is capable of filtering an output frequency to obtain a first resulting frequency and a second resulting frequency from the output frequency. Deconvolution device 216 may be coupled to any number of elements of system 200, including, but not limited to, detection device 202, detector 204, and/or frequency generator 206. In the exemplary embodiment of system 200 shown in FIG. 2A, deconvolution device is shown as being coupled to detection device 202.

Furthermore, and in an exemplary embodiment of a system 200 of the present disclosure, system 200 may further comprise a stimulator 218 capable of applying/exciting a current to detection device 202. An exemplary system 200 of the present disclosure may also comprise a data acquisition and processing system 220 capable of receiving conductance data from detector 204 and calculating parallel tissue conductance. In various embodiments of data acquisition and processing systems 220, data acquisition and processing systems 220 may be further capable of calculating a cross-sectional area of a luminal organ and/or determining plaque-type composition of a plaque within a luminal organ, based upon the conductance data. The data acquisition and processing systems 220 may also be capable of calculating vessel wall conductance, wall tissue thickness, parallel conductance, blood conductivity, total conductance, blood conductance, etc. or any of the parameters mentioned in the present application. Data acquisition and processing systems 220 of the present disclosure are considered to have a processor (processing means), memory, and a storage device (storage means) therein, such as a typical “computer” known in the art would have. Data acquisition and processing systems 220 of the present disclosure are therefore configured to receive data (such as conductance or impedance data) and process the same, such as being programmed to calculate parallel tissue conductance and/or cross-sectional area based on said conductance or impedance data.

In addition, an exemplary detection device 202 of the present disclosure may comprise any number of devices 202 as shown in FIGS. 2B-2G. Referring to FIGS. 2B, 2C, 2D, and 2E, several exemplary embodiments of the detection devices 202 are illustrated. The detection devices 202 shown contain, to a varying degree, different electrodes, number and optional balloon(s). With reference to the embodiment shown in FIG. 2B, there is shown an impedance catheter 20 (an exemplary detection device 202) with four electrodes 25, 26, 27 and 28 placed close to the tip 19 of the catheter 20. Proximal to these electrodes is an angiography or stenting balloon 30 capable of being used for treating stenosis. Electrodes 25 and 27 are excitation electrodes, while electrodes 26 and 28 are detection electrodes, which allow measurement of cross-sectional area during advancement of detection device 202, as described in further detail below. The portion of catheter 20 within balloon 30 includes an infusion port 35 and a pressure port 36.

In an exemplary embodiment, and shown in FIG. 2C, catheter 39 includes another set of excitation electrodes 40, 41 and detection electrodes 42, 43 located inside the angioplastic or stenting balloon 30 for accurate determination of the balloon cross-sectional area during angioplasty or stent deployment. These electrodes are in addition to electrodes 25, 26, 27 and 28.

In another exemplary embodiment, and as shown in FIG. 2G, several cross-sectional areas can be measured using an array of 5 or more electrodes. Here, the excitation electrodes 51, 52, are used to generate the current while detection electrodes 53, 54, 55, 56 and 57 are used to detect the current at their respective sites.

The tip of an exemplary catheter can be straight, curved or with an angle to facilitate insertion into the coronary arteries or other lumens, such as, for example, the biliary tract. The distance between the balloon and the electrodes is usually small, in the 0.5-2 cm range, but can be closer or further away, depending on the particular application or treatment involved.

In at least another embodiment, and shown in FIG. 2D, catheter 21 has one or more imaging or recording device, such as, for example, ultrasound transducers 50 for cross-sectional area and wall thickness measurements. As shown in this exemplary embodiment, transducers 50 are located near the distal tip 19 of catheter 21.

FIG. 2E shows an exemplary embodiment of an impedance catheter 22 without an angioplastic or stenting balloon. This catheter 22 also comprises an infusion or injection port 35 located proximal relative to the excitation electrode 25 and pressure port 36.

With reference to the exemplary embodiment shown in FIG. 2F, electrodes 25, 26, 27, 28 can also be built onto a wire 18, such as, for example, a pressure wire, and inserted through a guide catheter 23. Various wire 18 embodiments can be used separately (i.e., without a catheter), or can be used in connection with a guide catheter 37 as shown in FIG. 2E.

With reference to the embodiments shown in FIGS. 2B-2G, the impedance catheter advantageously includes optional ports 35, 36, 37 for suction of contents of the organ or infusion of fluid. Suction/infusion ports 35, 36, 37 can be placed as shown with the balloon or elsewhere both proximal or distal to the balloon on the various catheters.

In at least another embodiment (not illustrated), an exemplary catheter contains an extra channel for insertion of a guide wire to stiffen the flexible catheter during the insertion or data recording. In yet another embodiment (not illustrated), the catheter includes a sensor for measurement of the flow of fluid in the body organ.

As described below with reference to FIGS. 2H, 2I, and 2J, the excitation and detection electrodes are electrically connected to electrically conductive leads in the catheter for connecting the electrodes to the stimulator 218, for example.

FIGS. 2H and 2I illustrate two exemplary embodiments 20A and 20B of the catheter in cross-section. Each embodiment has a lumen 60 for inflating and deflating a balloon and a lumen 61 for suction and infusion. The sizes of these lumens can vary in size. The impedance electrode electrical leads 70A are embedded in the material of the catheter in the embodiment in FIG. 2H, whereas the electrode electrical leads 70B are tunneled through a lumen 71 formed within the body of catheter 70B in FIG. 2I.

Pressure conduits for perfusion manometry connect the pressure ports 90, 91 to transducers included in system 200. As shown in FIG. 2H, pressure conduits 95A may be formed in 20A. In another exemplary embodiment, shown in FIG. 2I, pressure conduits 95B constitute individual conduits within a tunnel 96 formed in catheter 20B. In the embodiment described above where miniature pressure transducers are carried by the catheter, electrical conductors will be substituted for these pressure conduits.

At least a portion of an system for obtaining a parallel tissue conductance within a luminal organ of the present disclosure is shown in FIG. 2J. As shown in FIG. 2J, an exemplary system 200 of the present disclosure comprises a detection device operably connected to a manual or automatic system 222 for distension of a balloon and to a system 224 for infusion of fluid or suction of blood. The fluid, in an exemplary embodiment, may be heated to 37-39° C. or equivalent to body temperature with heating unit 226. In addition, and as shown in FIG. 2J, system 200 may comprise a stimulator 218 to provide a current to excite detection device 202, and a data acquisition and processing system 220 to process conductance data. Furthermore, an exemplary system 200 may also comprise a signal amplifier/conditioner (not shown) and a computer 228 for additional data processing as desired. Such a system 200 may also optionally contain signal conditioning equipment for recording of fluid flow in the organ.

In an exemplary embodiment, the system 200 is pre-calibrated and the detection device 202 is available in a package. The parallel conductance, CSA, plaque-type, and other relevant measures such as distensibility, tension, etc., may then typically appear on the display of computer 228. In such an embodiment, the user can then remove the stenosis by distension or by placement of a stent.

If more than one CSA is measured, for example, system 200 can also contain a multiplexer unit or a switch between CSA channels. In at least one embodiment, each CSA measurement will be through separate amplifier units. The same may account for the pressure channels as well.

In at least one embodiment, the impedance and pressure data are analog signals which are converted by analog-to-digital converters 230 and transmitted to a computer 228 for on-line display, on-line analysis and storage. In another embodiment, all data handling is done on an entirely analog basis. The analysis may also includes software programs for reducing the error due to conductance of current in the organ wall and surrounding tissue and for displaying the 2D or 3D-geometry of the CSA distribution along the length of the vessel along with the pressure gradient. In an exemplary embodiment of the software, a finite element approach or a finite difference approach is used to derive the CSA of the organ stenosis taking parameters such as conductivities of the fluid in the organ and of the organ wall and surrounding tissue into consideration. In another embodiment, the software contains the code for reducing the error in luminal CSA measurement by analyzing signals during interventions such as infusion of a fluid into the organ or by changing the amplitude or frequency of the current from the constant current amplifier. The software chosen for a particular application, preferably allows computation of the CSA with only a small error instantly or within acceptable time during the medical procedure.

### EXEMPLARY METHOD

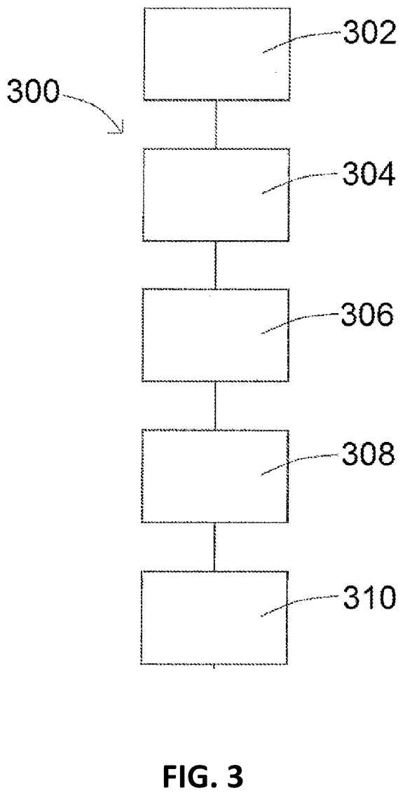

Steps of an exemplary two frequency method of the present disclosure are shown in FIG. 3. As shown in FIG. 3, an exemplary method 300 comprises the step of introducing at least part of a detection device 202 into a luminal organ at a first location (introduction step 302), whereby detection device 202 comprises a detector 204, and applying current to detection device 202 to allow detector 204 to operate (current application step 304). The application/excitation of current may be performed using a stimulator 218. Method 300, in at least one embodiment, further comprises the steps of introducing a first signal having a first frequency and a second signal having a second frequency through detection device 202 (frequency introduction step 306). In an exemplary embodiment of a method 300 of the present disclosure, frequency introduction step 306 is performed using a frequency generator 206. In an exemplary embodiment, only a first frequency and a second frequency are the only frequencies introduced.

Exemplary method 300 further comprises the step of measuring an output conductance of the first signal and the second signal at the first location (conductance measurement step 308), and the step of calculating a parallel tissue conductance at the first location (calculation step 310), in an exemplary embodiment, based in part upon the output conductance and the conductivity of the injected solution.

Calculation step 310, in at least one embodiment, may comprise the step of calculating a diameter or cross-sectional area of the luminal organ at the first location. In an exemplary embodiment wherein the first location comprises a plaque site, calculation step 312 may comprise the step of determining plaque-type composition of a plaque at the plaque site. Calculation step may also comprise the step of determining vessel wall conductance, wall tissue thickness, parallel conductance, blood conductivity, total conductance, blood conductance, etc. or any of the parameters mentioned in the present application.

Conductance measurement step 308 may include the measurement of an output conductance whereby the output conductance comprises a first conductance value and a second conductance value. In at least one embodiment, the first conductance value corresponds to the first frequency and the second conductance value corresponds to the second frequency. In an exemplary embodiment, calculation step 310 may comprise the step of deconvoluting the output conductance to obtain a first conductance value and a second conductance value from the output conductance. In at least one embodiment, the step of deconvoluting the output conductance is performed using a deconvolution device 216.

In at least one embodiment of a method 300 of the present disclosure, the output conductance comprises a mixed signal. In such an embodiment, calculation step 310 may further comprise the step of deconvoluting the mixed signal to obtain a first conductance value and a second conductance value from the mixed signal.

Frequency introduction step 306 may involve the introduction of signals having frequencies with various characteristics. For example, and in at least one embodiment, the first signal and the second signal may be repeatedly alternated to form a multiplexed signal. The alternated signals may then be separated in time by a short amount of time, for example 1 to 1000 milliseconds. In an exemplary embodiment, the first signal and the second signal are separated in time by less than 100 milliseconds. In another exemplary embodiment, the first signal and the second signal are separated in time by less than 10 milliseconds. Frequency introduction step 306 may also involve the introduction of signals whereby the first signal and the second signal are combined to form a combined signal.

In an exemplary embodiment of conductance measurement step 308 of an exemplary method 300 of the present disclosure, conductance measurement step 308 may be performed using an exemplary detection device 202. In at least one embodiment of a detection device 202 used in connection with a method 300 of the present disclosure, detector 204 of detection device 202 comprises detection electrodes 26, 28 positioned in between excitation electrodes 25, 27, wherein excitation electrodes 25, 27 are capable of producing an electrical field.

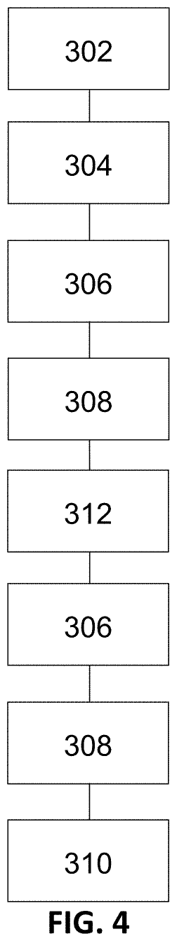

In at least another exemplary embodiment of a method 300 of the present disclosure, and as shown in FIG. 4, method 300 comprises introduction step 302, current application step 304, and frequency introduction step 306 as referenced above. This additional exemplary method 300 then comprises the step of measuring an output conductance of a first signal and a second signal at the first location (conductance measurement step 308), whereby conductance measurement step 308 involves, in such an embodiment, measuring a first output conductance at the first location within a luminal organ in connection with a fluid native to the first location, with the native fluid having a first conductivity. After the foregoing conductance measurement step 308 has been performed, the step of moving the device to a second location 312 may be performed. At the second location, a second frequency introduction step 306 may be performed and a second conductance measurement step 308 may be performed to measure a second output conductance of the first signal and the second signal at the second location. With this acquired information, an exemplary method 300 of the present disclosure may include the step of calculating a parallel tissue conductance at the first location (calculation step 310) and the second location. In such an exemplary embodiment, based on the results of the calculations, a lumen profile may be generated.

In another embodiment, calculations utilizing the measurements obtained from the first location may be performed before moving the device to the second location. In another embodiment, the device may be moved to any number of locations wherein the frequency introduction step and the conductance measurement step are performed at each location; and the calculation step performed for the measurements obtained from each location to generate a profile. In another embodiment, the device is moved continuously along a length and the frequency introduction step and conductance measurement step is performed while the device is in motion. The frequency introduction step and the conductance measurement step may be performed a plurality of times while the device is in motion, and the calculation step performed for each measurement obtained so that a lumen profile may be generated.

Various characteristics of the aforementioned signals, generating the same, conductance values, filtering, frequencies, output signals, etc., apply to any number of methods 300 referenced herein. For example, and as shown in FIG. 4, calculation step 310 of method 300 may comprise the step of deconvoluting the second output conductance to obtain a first resulting conductance value and a second resulting conductance value from the second output conductance as referenced above in connection with method 300 shown in FIG. 3.

In addition, calculation step 310, in at least one embodiment, may comprise the step of calculating a cross-sectional area of the luminal organ at the first location. In an exemplary embodiment wherein the first location comprises a plaque site, calculation step 312 may comprise the step of determining plaque-type composition of a plaque at the plaque site.

To consider a method of obtaining conductance values and related impedance, which are used to determine CSA or evaluate the type and/or composition of a plaque, a number of approaches may be used. In one approach, luminal cross-sectional area is measured by introducing a catheter from an exteriorly accessible opening (e.g., mouth, nose or anus for GI applications; or e.g., mouth or nose for airway applications) into the hollow system or targeted luminal organ. In an exemplary approach, conductance is measured by introducing a catheter from an exteriorly accessible opening into the hollow system or targeted luminal organ. For cardiovascular applications, the catheter can be inserted into the organs in various ways, for example, similar to conventional angioplasty. In at least one embodiment, an 18 gauge needle is inserted into the femoral artery followed by an introducer, and a guide wire is then inserted into the introducer and advanced into the lumen of the femoral artery. A 4 or 5 Fr conductance catheter is then inserted into the femoral artery via wire and the wire is subsequently retracted. The catheter tip containing the conductance (excitation) electrodes can then be advanced to the region of interest by use of x-ray (using fluoroscopy, for example). In another approach, this methodology is used on small to medium size vessels, such as femoral, coronary, carotid, and iliac arteries, for example.

### MATHEMATICAL MODELING

Mathematical modelling of this technique is described as follows.

The electric field distribution in the lumen and the surrounding tissue is inhomogeneous which makes the conductance-diameter relationship nonlinear. The electric field intensity varies with distance from the center of the lumen. When the diameter increases, the far-field electric field contribution to the sensing electrodes decreases and hence the measured conductance approaches a saturation value. On the other hand, if the radius of the lumen decreases, the electric field distribution tends to be more homogeneous, so the conductance-diameter relationship is more linear.

Since the electric field generated by source electrodes is not homogeneous and the electric field intensity inside the lumen is not constant, the blood conductance can be obtained by solving Laplace's equation which results in the dependence of G_{b} (real conductance) as a function of lumen diameter as follows:

$${G}_{b} = \frac{{πσ}_{bReal} d ( {d}^{2} - {L}^{2} )}{4 L} ( \frac{2}{\sqrt{{d}_{GW}^{2} + {d}^{2}}} - \frac{2}{\sqrt{{d}_{b}^{2} + {d}^{2}}} ) \qquad ( 1 )$$

where d is the excitation electrode separation distance, L is the sensing electrode separation distance, σ_{bReal} is the blood conductivity, d_{GW} is the diameter of the guidewire, and d_{b} is the lumen diameter. This equation assumes that the radius of the sensing electrodes is small enough that their influences on the electric field distribution are negligible. It is important to note that σ_{bReal} defined as real conductivity can be obtained by equation (1) since all the elements of equation (1) can be measured to obtain σ_{bReal}. In the literature, the value of conductivity has been obtained from the following equation which is only valid for a homogeneous field distribution: For a homogeneous field distribution, equation (1) can be simplified to the following equation:

$${G}_{b} = \frac{{πσ}_{bIdeal} ( {d}_{b}^{2} - {d}_{GW}^{2} )}{4 L} \qquad ( 2 )$$

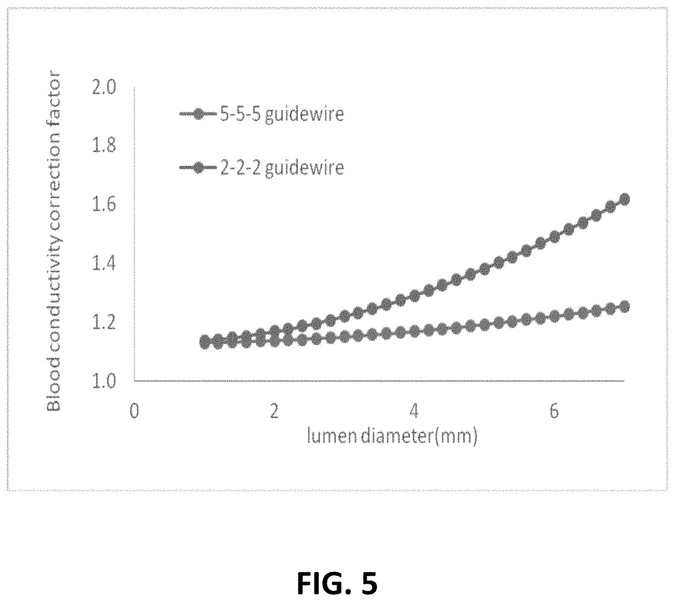

The conductivity of the equation (2) is called ideal conductivity since it represents the ideal conductance equation which may be an approximation to reality. The ratio of real to ideal conductivities is defined as σ_{bReal}/σ_{bIdeal} and it has been plotted as function of the lumen diameter in FIG. 5 for the two different guidewires (the 5-5-5 guidewire represented by the lower line and the 2-2-2 guidewire represented by the upper line). It is clear from FIG. 5 that the real conductivity is higher than the idealized one using the same value for the conductance and this difference increases with lumen diameter. In other words, if one wants to use equation (2) for conductance, the measured real conductivity should be divided by the σ_{bReal}/σ_{bIdeal} which is designated as Blood Conductivity Correction Factor (BCCF), to find the ideal conductivity.

This is also true for the vessel wall where both real and ideal conductivities are defined. The vessel wall conductance is the same form as equation (1) but with different parameters as follows:

$${G}_{t} = \frac{{πσ}_{tReal} d ( {d}^{2} - {L}^{2} )}{4 L} ( \frac{2}{\sqrt{{d}_{b}^{2} + {d}^{2}}} - \frac{2}{\sqrt{{d}_{t}^{2} + {d}^{2}}} ) \qquad ( 3 )$$

The new parameters in this equation are σ_{tReal} and dₜ. The σ_{tReal} is defined as tissue wall real conductivity and dₜ is the outside diameter of the vessel wall. The dₜ and d_{b} are related by the following equation:

dₜ=d_{b}+2t (4)

where t is the tissue thickness of the vessel wall. The ratio of the wall tissue thickness and lumen diameter is defined as TR:

TR=t/d_{b} (5)

dₜ=d_{b}(1+2TR) (6)

The ideal tissue conductivity, σ_{tIdeal}, is defined by the following equation:

$${G}_{t} = \frac{{πσ}_{tIdeal} ( {d}_{t}^{2} - {d}_{b}^{2} )}{4 L} \qquad ( 7 )$$

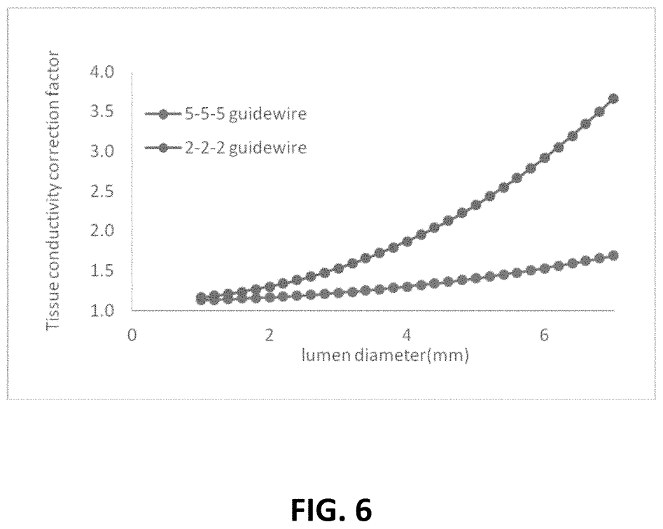

If equation (7) is used for the vessel wall tissue conductance calculation, ideal tissue conductivity should be used. This will be obtained by dividing real tissue conductivity by the vessel wall Tissue Conductivity Correction Factor (TCCF) which is a function of lumen diameter, guidewire diameter, and TR which can be obtained from FIG. 6 (with the 5-5-5 guidewire represented by the lower line and the 2-2-2 guidewire represented by the upper line) adjusted for the value of TR. TCCF is defined as the ratio of real to ideal tissue conductivity.

Blood is considered a heterogeneous medium because of the erythrocytes in plasma. Often the electrical characteristics of suspensions of blood are modeled using the well-known three-element model¹⁵. In this lumped model, one resistor represents the electrical resistance of plasma, while the effect of the cell membrane capacitance of the erythrocytes is modeled by a capacitor. Furthermore, another resistor represents the effect of the interior cell resistance of the erythrocytes. The admittance in three frequencies to model blood should also be measured. Our analysis shows that the results are similar in the frequency range of our interest if it is replaced with two-frequency model where the effect of the interior cell resistance is neglected. The two-frequency model was adopted for the sake of simplicity. The behavior of blood is therefore modeled in the frequency domain by a RC circuit where R represents the blood resistance and C represents the capacitance across the red blood cell membrane. Blood impedance at two different frequencies needs to be measured to derive the values of R and C.

The blood electrical conductivity remains not only a function of frequency, but it is also a function of diameter as evidenced by the in vitro experimental results which will be discussed later. This is due to several phenomena that occur on the interface between the electrode and the blood layer which acts like a capacitor with charge transfer and polarization resistance¹⁶. This phenomenon was also observed in the case where the electrolyte was NaCl solution where there is no effect of the red blood cell interior resistance and its membrane acts like a capacitor. This is due to the electrolyte ions motion which increases with frequency and thus results in increase of electrical conductivity¹⁷.

A two-frequency model can be best represented by a classical parallel RC-circuit to calculate the blood electrical conductivity as function of frequency. The value of the resistance, R, and capacitance, C, are a function of impedance and frequency as follows:

$$R = {[ \frac{{( {Z}_{1} {Z}_{2} {ω}_{2} )}^{2} - {( {Z}_{1} {Z}_{2} {ω}_{1} )}^{2}}{{( {Z}_{2} {ω}_{2} )}^{2} - {( {Z}_{1} {ω}_{1} )}^{2}} ]}^{1 / 2} \qquad ( 8 )$$

$$C = {[ \frac{{R}^{2} - {Z}_{2}^{2}}{{( {Z}_{2} {ω}_{2} R )}^{2}} ]}^{1 / 2} \qquad ( 9 )$$

where ωᵢ=2·π·ƒᵢ, Z₁ and Z₂ are the impedances at frequencies ƒ₁ and ƒ₂, which are the ratio of the measured voltages to the applied current. In the two-frequency model, the value of R represents the total resistance of the combined system which includes both the blood in the lumen and the vessel wall. In the two-frequency model, the inverse R is the total conductance of system.

A goal of this studies referenced herein is to develop a model to predict the parallel conductance. The measured total conductance in an infinite medium, G_{inf}, between two sensing electrodes can be approximated by the following equation¹⁴.

$${G}_{inf , bath} = \frac{{πσ}_{bath} d ( {d}^{2} - {L}^{2} )}{4 L} ( \frac{2}{\sqrt{{d}_{GW}^{2} + {d}^{2}}} ) \qquad ( 10 )$$

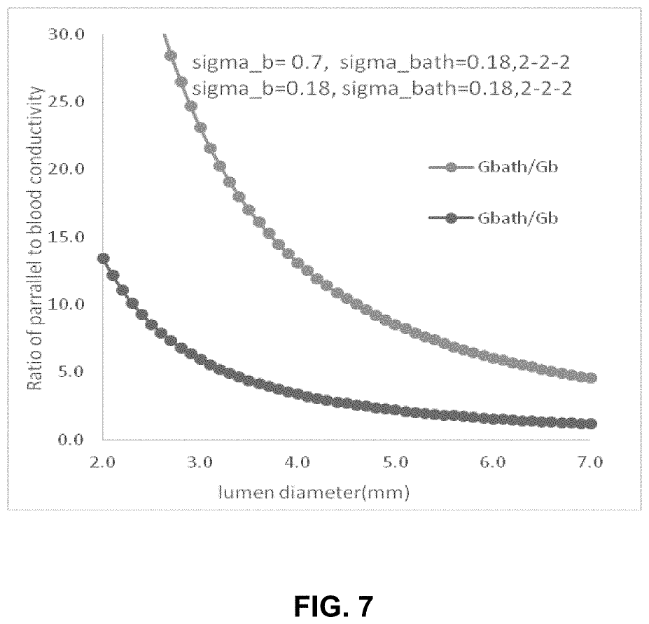

The parallel conductance for an infinite medium can be obtained from this equation by replacing d_{b} for d_{GW} since anything beyond the lumen diameter is parallel conductance. The ratio of the parallel conductance for an infinite tissue thickness to the conductance inside of the lumen, is plotted in FIG. 7 (the upper line) as function of lumen diameter for 2-2-2 guidewire, assuming that both the blood conductivity and the bath conductivity are the same. This graph demonstrates that the conductance inside the lumen is much smaller than the parallel conductance. Two factors are helpful to increase the share of the lumen conductance. One is that the blood conductivity is about 4-5 larger than the surrounding tissue conductivity and second is that the surrounding thickness is finite. FIG. 7 also shows the ratio with blood conductivity of 0.7 S/m (the lower line), where the lumen conductance contribution is much larger. For a finite surrounding tissue thickness, the parallel conductance is a function of surrounding tissue conductivity, electrical field distribution across the tissue and its thickness. The following model is proposed for the parallel conductance, G_{bath}, for a finite thickness of the surrounding tissue:

$${G}_{bath} = \frac{{πσ}_{bath} d ( {d}^{2} - {L}^{2} )}{4 L} ( \frac{2}{\sqrt{{d}_{t}^{2} + {d}^{2}}} - \frac{2}{\sqrt{{d}_{bath}^{2} + {d}^{2}}} ) \qquad ( 11 )$$

where σ_{bath} is the surrounding tissue conductivity and d_{bath} is the surrounding tissue thickness. An estimate of the surrounding tissue thickness is sufficient for the accurate determination of the parallel conductance since the electrical field drops rather abruptly from the center of the lumen and it is even less sensitive for the larger diameters of the lumen which is the region of interest for peripheral vessels. The tests results and comparison with the model are discussed in the result section.

### Methods

The general method to obtain the lumen and vessel wall tissue conductivity, and the method for the ex vivo study, are described herein. Finally, both 5-5-5 and 2-2-2 spacing wires were used in the experiments to measure the diameters of peripheral (4-10 mm diameter range) and coronary (2-5 mm diameter range), respectively. Said devices, as well as devices disclosed within International Patent Application Serial No. PCT/US2017/045581, filed Aug. 4, 2017, the contents of which are incorporated herein directly and by reference, can be used to perform any number of methods of the present disclosure. The spacing numbers represent the distance between the four electrodes in mm. The larger spacing is associated with the 035″ diameter guidewire for peripheral vessels while the smaller one is associated with the 014″ diameter guidewire for coronary vessels. This is to ensure that the excitation to excitation distances, d (18 mm and 9 mm for 5-5-5 and 2-2-2, respectively) is approximately twice the diameter (d_{b}) of the largest vessel of interest (10 and 4 for peripheral and coronary, respectively; i.e., d/d_{b}≥2) to obey the cylindricity assumptions.

### Phantom Studies

The blood and lumen wall tissue conductivities are needed to estimate the lumen diameter by the injection-less method. The ideal method to determine the blood conductivity in the lumen is in phantoms where the diameter is known and the surrounding tissue conductivity is zero. The blood conductance was estimated using the two-frequency approach. The admittance at various frequencies was calculated by applying the values obtained from equations 8 and 9 for frequencies of 10 and 100 kHz. The blood conductivities were calculated from the value (system resistance) determined from the two-frequency model. The results are utilized in the next section.

### Ex Vivo Studies

An objective of the ex vivo study was to estimate the mean and standard deviation (SD) of the difference in vessel diameter as measured by an optical method and the model prediction for different lumen sizes. The mean and SD of the difference in diameter was calculated based on 11 experiments of different vessel diameters. An estimate of the vessel wall electrical conductivity is needed to predict the vessel diameter using the two-frequency approach. The bovine carotid artery was placed inde-ionized water bath perfused with the 0.45% saline at room temperature to measure the total impedance of tissue. The lumen diameter and the tissue thickness were measured optically. A two-frequency model was used to convert the measured voltage to the sum conductance of the lumen and the tissue wall. The lumen conductance can be obtained from the known diameter of the lumen and the conductivity of the saline solution which was obtained from the phantom experiments. The tissue conductance can be determined as the total minus the lumen conductance. The tissue electrical conductivity was calculated from the tissue conductance and the cross sectional area of the tissue. Experiments were performed with bovine carotid artery to estimate the electrical conductivity of the tissue surrounding the artery at room temperature. The artery diameter was measured optically to be about 3.2 mm with the thickness of the vessel wall tissue of about 2.5 mm at no-load state (zero pressure condition). The excitation current was 100 μArms and the voltages were measured in the frequency range of 10-80 kHz.

Once the tissue conductivity is known, it can be used to analyze the mean differences in diameter as measured by the optical method and the model prediction for different lumen sizes as follows. The total conductance can be separated into two components, lumen conductance, G_{b} and parallel conductance, G_{P}. The following equation holds for the two conductances:

G=G_{b}+G_{P}=1/R (12)

The lumen diameter can be obtained by combining equations (2), (7), and (12) to yield following relation:

$${d}_{b =} \sqrt{\frac{4 L + π R {σ}_{bIdeal} {d}_{GW}^{2}}{π R ( {σ}_{bIdeal} + 4 {σ}_{tIdeal} TR ( 1 + TR ) )}} \qquad ( 13 )$$

where d is the excitation electrode separation distance, L is the sensing electrode separation distance, σ_{bIdeal} and σ_{tIdeal} are the blood and tissue conductivities, respectively; d_{GW} is the diameter of the guidewire, R is the system resistance defined in the two frequency model, TR is the ratio of the wall tissue thickness and lumen diameter, and d_{b} is the lumen diameter. The lumen diameter can be estimated from the total conductance, and the conductivities of the blood and the vessel wall tissue. The blood conductivity is both a function of frequency and lumen diameter. An iterative method was used to calculate the lumen diameter. A two-frequency model was used to determine the R resistance of the total system by measuring the voltages at two frequencies.

A series of ex vivo experiments were performed to determine the ratio of lumen conductance to parallel conductance. The tests were performed with the bovine carotid artery immersed in a 0.1% NaCl solution bath to simulate the level of parallel conductance in vivo. The width or diameter of the bath around the vessel was varied in four dimensions of 2.1, 4.9, 11.7 and 20.9 mm. The 0.45% saline solution was used to perfuse through the vessel lumen. The guidewire had electrode spacings of 5-5-5 and the applied current was 300 μA rms. At each step, the voltages were measured at two frequencies. The first experiment was performed without any parallel conductance (suspended in air) to calculate the vessel wall tissue conductivity.

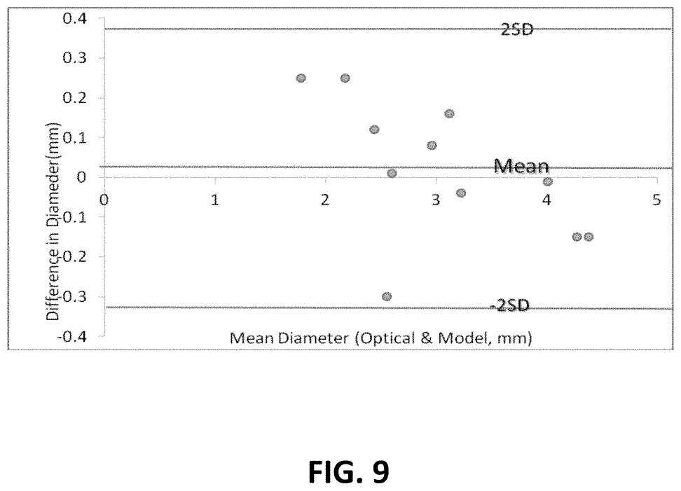

The methodology for the determination of vessel diameter using the two-frequency method in vivo will be an iterative process as follows:
1) Blood conductivity: For ex vivo conditions, the description of blood conductivity measurements in phantoms is described above. Under in vivo conditions, the guidewire will be inserted in the standard introducing catheter (typically 5 Fr or 6 Fr) with some aspiration of subject blood into the catheter to measure blood voltage drops across the detection electrodes at 10 and 20 kHz. The value of voltage differences at these two frequencies are large enough for small lumen diameters. The voltages will be converted to the impedance by dividing the electrical current to the measured voltages. The blood conductance in the catheter will be calculated from equation (8), which is the inverse of R. The blood ideal conductivity in the catheter will be calculated since the diameter of the introducer catheter is known.
2) Total conductance: The sizing guidewire will then be inserted in the lumen of blood vessel to measure blood voltage drops across the detection electrodes at 10 and 20 KHz. The voltages will be converted to the impedance by dividing the electrical current to the measured voltages. The total conductance, G, in the lumen will be calculated from equation (8) which is the inverse of R.
3) Blood conductance: Equation (2) will be applied to determine the blood conductance, G_{b}, using the blood conductivity in the lumen obtained in step 1. This term is only a function of the lumen diameter which is the variable of interest. The blood conductance will be calculated by an initial estimate of the lumen diameter.
4) Parallel conductance: It can be determined from equation (12) by G−G_{b}, which is only function of lumen diameter. This is based on assumption that the tissue wall conductance makes up a portion of the parallel conductance. The parallel conductance can also be obtained by the integration of a term from the lumen wall to the surrounding tissue thickness. This term is a product of the tissue conductivity, the electric field at any point within the surrounding tissue thickness and the corresponding annular surface area. The value of the parallel conductance from this calculation is also a function of the lumen diameter since the integration starts from the lumen wall. The parallel conductance has been modelled and discussed in the next section. The model results indicate that there is a fixed ratio of the parallel conductance to total conductance for a specific guidewire, specific blood, specific diameter, specific surrounding tissue conductivity and specific surrounding tissue thickness. Any resulting error associated with the parallel conductance calculation will result in a similar error in the lumen conductance calculation, which in turn results in an error in the lumen diameter calculation. The error in the lumen diameter is almost half the error in the lumen conductance for small errors since the lumen diameter is proportional to the square root of lumen conductance.
5) Diameter determination: The diameter can be determined by setting the values of the parallel conductance determined by the two methods to be equal. If this value of the lumen diameter is not the same as the initial estimate, then a new value will be selected and the process will be repeated till the parallel conductance from the two methods approach each other within 2%. This method is shown in the flow chart of FIG. 9.

### RESULTS

### Phantom Experiments

The experiments were performed at room temperature with both blood and 0.45%. NaCl saline solution in phantoms of various diameters with 5-5-5 and 2-2-2 guidewires from 0.1 to 100 KHz with 100 μA rms current of sinusoidal shape. The measured voltages are shown in Table 1, shown below, for the 0.45% saline solution for 5-5-5 guidewire at room temperature.

### Saline 0.45%

TABLE 1
Measured voltages, mV, as function of phantom diameters and frequencies
for .45% saline solution at room temperature for 5-5-5 guidewire.

| Voltage (mV) | Diameter (mm) | | | | | |
|---|---|---|---|---|---|---|
| (mV) | 1.75 | 3 | 4 | 6 | 8 | (Nominal Dia (mm)) |
| | 1.75 | 2.97 | 3.96 | 6.00 | 8.00 | (Actual Dia (mm)) |
| Freq (kHz) | 1.5074 | 2.83 | 3.86 | 5.93 | 7.95 | (Effective Dia (mm)) |
| 1 | 259.00 | 61.00 | 55.00 | 25.60 | 15.60 | |
| 10 | 209.00 | 57.00 | 52.00 | 24.30 | 14.90 | |
| 20 | 176.00 | 55.00 | 48.00 | 23.50 | 14.50 | |
| 40 | 125.00 | 50.00 | 43.00 | 21.70 | 14.00 | |
| 60 | 90.00 | 44.00 | 38.00 | 19.40 | 13.00 | |
| 80 | 69.00 | 39.00 | 33.00 | 17.50 | 12.00 | |
| 100 | 55.00 | 34.00 | 30.00 | 16.00 | 11.00 | |

The ideal conductivities have been calculated from the R resistance values determined from the two-frequency model. Example of R values is shown in Table 2, included below, for lumen diameter of 4 mm with 0.45% saline solution at room temperature at different frequencies.

TABLE 2
An example of R and C values for a 4 mm lumen diameter with 0.45%
saline solution at room temperature for 5-5-5 guidewire. The frequency
range is 1-80 KHz. Volt1 and volt2 are measured voltages at the
corresponding frequencies. db is the calculated diameter.

| freq1 | freq2 | volt1 | volt2 | R | C | db |
|---|---|---|---|---|---|---|
| 1,000 | 10,000 | 55 | 52 | 5.50E+02 | 1.00E−08 | 4.0056 |
| 1,000 | 20,000 | 55 | 48 | 5.50E+02 | 8.10E−09 | 4.0060 |
| 1,000 | 40,000 | 55 | 43 | 5.50E+02 | 5.77E−09 | 4.0064 |
| 1,000 | 60,000 | 55 | 38 | 5.50E+02 | 5.05E−09 | 4.0064 |
| 1,000 | 80,000 | 55 | 33 | 5.50E+02 | 4.82E−09 | 4.0065 |
| 10,000 | 20,000 | 52 | 48 | 5.36E+02 | 7.36E−09 | 3.9750 |
| 10,000 | 40,000 | 52 | 43 | 5.28E+02 | 5.37E−09 | 4.0018 |
| 10,000 | 60,000 | 52 | 38 | 5.27E+02 | 4.83E−09 | 4.0076 |
| 10,000 | 80,000 | 52 | 33 | 5.26E+02 | 4.70E−09 | 4.0089 |
| 20,000 | 40,000 | 48 | 43 | 5.01E+02 | 4.75E−09 | 4.0049 |
| 20,000 | 60,000 | 48 | 38 | 4.99B+02 | 4.52E−09 | 4.0127 |
| 20,000 | 80,000 | 48 | 33 | 4.99E+02 | 4.52E−09 | 4.0128 |
| 40,000 | 60,000 | 43 | 38 | 4.88E+02 | 4.38E−09 | 3.9976 |
| 40,000 | 80,000 | 43 | 33 | 4.91E+02 | 4.46E−09 | 3.9875 |
| 60,000 | 80,000 | 38 | 33 | 4.99E+02 | 4.52E−09 | 3.9399 |

The R values have been calculated for all combinations of two frequencies. These combinations have been grouped into five categories. In each category, the first frequency is the same while the second frequency was made to vary. It is observed that the value of R (inverse of conductance) is relatively constant in each group and decreases with increase of frequency. This results in increase of conductivity with frequency. The ideal conductivities are calculated from R values and lumen diameters and are shown in Table 3, noted below.

TABLE 3
Calculated ideal conductivity of 0.45% saline solution
for various phantom diameters as function of frequency
at room temperature for 5-5-5-guidewire.

| Conductivity (σ) | Diameter (mm) | | | | |
|---|---|---|---|---|---|
| (S/m) | 1.75 | 3 | 4 | 6 | 8 |
| Freq (Hz) | | | | | |
| 10000 | 1.7000 | 1.6000 | 0.9500 | 0.8800 | 0.8000 |
| 20000 | 2.2000 | 1.6000 | 1.0000 | 0.9000 | 0.8100 |
| 40000 | 2.4000 | 1.6000 | 1.0300 | 0.9200 | 0.8200 |
| 60000 | 2.6000 | 1.7000 | 1.0400 | 0.9500 | 0.8300 |
| 80000 | 2.8000 | 1.8000 | 1.0600 | 0.9500 | 0.8300 |

The conductivities decrease with diameter and increase with frequency. The same procedure was performed with 2-2-2 guidewire and the ideal conductivities results are shown in Table 4, noted below, for 0.45% saline solution.

TABLE 4
Calculation of 0.45% saline solution ideal conductivity,
S/m, for various phantom diameters and frequency
for 2-2-2 guidewire at room temperature.

| | |
|---|---|
| Conductivity | Diameter (mm) |

| | | | | |
|---|---|---|---|---|
| (S/m) | 4.0 | 3.30 | 2.50 | 1.75 |
| Freq (kHz) | | | | |
| 10 | 1.1300 | 1.4000 | 1.4400 | 1.9000 |
| 20 | 1.1800 | 1.4500 | 1.4800 | 1.9500 |
| 40 | 1.2100 | 1.5000 | 1.5000 | 1.9700 |
| 60 | 1.2200 | 1.6000 | 1.6000 | 2.0300 |
| 80 | 1.2300 | 1.7000 | 1.6500 | 2.0500 |

### Ex Vivo Experiments

Table 5, noted below, shows the tissue wall conductivities at various frequencies where the lumen diameter is 3.2 mm with wall thickness of 2.5 mm (TR was calculated to be 0.79).

TABLE 5
Vessel wall tissue conductivities at various frequencies at room
temperature for 0.45% saline solution flowing inside the lumen

| | Diameter (mm) | | | | |
|---|---|---|---|---|---|
| | σ_{bIdeal} | | | | |
| Freq (Hz) | d = 3.2 mm | GL | Gt | dt | σ_{tIdeal} |
| 10 | 1.4 | 1.75 | 2.97 | 8.31 | 0.39 |
| 20 | 1.5 | 1.88 | 2.98 | 8.31 | 0.39 |
| 40 | 1.4 | 1.75 | 3.12 | 8.31 | 0.41 |
| 60 | 1.5 | 1.88 | 3.04 | 8.31 | 0.40 |
| 80 | 1.6 | 2.01 | 3.16 | 8.31 | 0.41 |

The tests were performed at room temperature with 5-5-5 guidewire with 0.45% saline solution flowing inside the lumen. The results indicate that tissue conductivity is essentially constant in this frequency range.

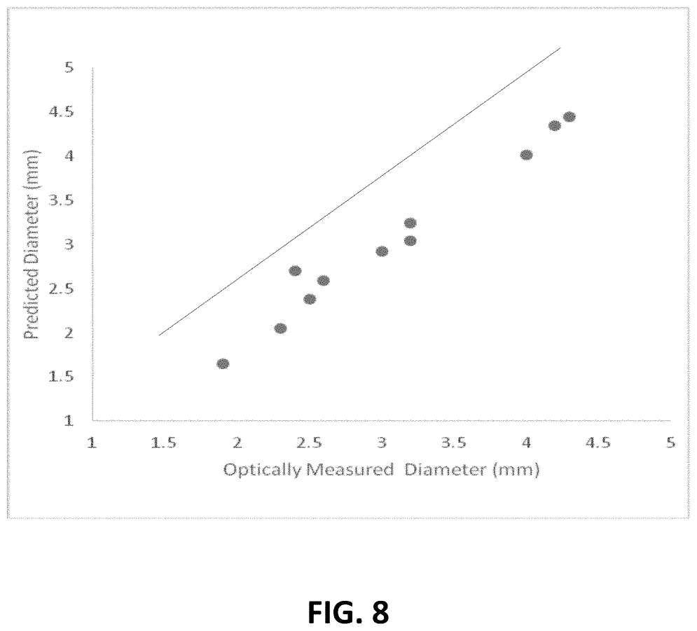

Eleven ex vivo experiments were performed to validate the model for both 5-5-5 and 2-2-2 guidewires with 0.45% saline solution flowing inside the lumen. The lumen diameters ranged from 1.7 mm to 8 mm (TR range of 0.16 to 0.9). The tissue conductivity for all the tests data analysis were set to be 0.4 S/m according to Table 5. The saline solution conductivities were obtained from the conductivities tables by iteration to correspond to the final lumen diameter prediction. The results are shown in FIG. 8 with line of equality. The figure compares the diameters obtained by the optical measurement and the model prediction for the 11 experiments. The Bland-Altman plot is shown in FIG. 9. The abscissa of this chart represents average diameter obtained from optical measurement and model prediction for the 11 experiments. The ordinate of this chart represents the difference in these two diameters for the 11 data points. One standard deviation (1SD) was 0.17 mm and the majority of the data (70%) fall within 1SD and all the data fall within 2SDs. The results indicate that the two-frequency model can accurately predict the lumen diameter in ex vivo measurements.

Table 6, shown below, shows the variation of total conductance with respect to bath width.

TABLE 6
Variation of total conductance with respect to the bath thickness.

| G_total | t_bath | | | | |
|---|---|---|---|---|---|
| Freq (kHz) | 0.000 | 2.118 | 4.868 | 11.668 | 20.868 |
| 10 | 4.6579 | 6.4886 | 8.3578 | 11.3277 | 12.8155 |
| 20 | 4.6579 | 6.6399 | 8.6467 | 11.9102 | 13.4538 |
| 40 | 4.7826 | 6.6144 | 7.9449 | 11.6696 | 12.4735 |

The parallel conductance increases with the bath width since the increase in the total conductance is only due to the bath width. Table 7, shown below, demonstrates the ratio of bath conductance to the total conductance as function of the bath width at different frequencies for the same vessel.

TABLE 7
Ratio of experimental values of parallel conductance to the total conductance
as function of the bath thickness at different frequencies, TR = 0.79.

| % (G_bath/G_total(r_bath_MAX)) | t_bath (mm) | | | |
|---|---|---|---|---|
| Freq (kHz) | 2.118 | 4.868 | 11.668 | 20.868 |
| 10 | 14.29% | 28.87% | 52.04% | 63.65% |
| 20 | 14.73% | 29.65% | 53.91% | 65.38% |
| 40 | 14.68% | 25.35% | 55.21% | 61.66% |

The ratio is defined as the ratio of bath conductance at specified bath width to the total conductance at the width of 20.9 mm (maximum width). The table was constructed by subtracting lumen and vessel wall tissue conductance from the total conductance at each specified bath width. Table 7 indicates that about 65% of the total conductance goes to the bath. This value is for very thick vessel wall since TR is about 0.8 (bovine vessel). The parallel conductance increases if TR is reduced. Table 8, shown below, shows the results of the variation of ratio of the parallel conductance to the total conductance with respect to TR predicted by the model. For example, this ratio increases to 82% when TR is reduced to 0.3. This corresponds to wall thickness of about 1 mm for this vessel with diameter of 3.2 mm.

TABLE 8
Variation of the ratio of parallel conductance to the total
conductance with respect to TR, predicted by the model.

| | t_bath | | | | |
|---|---|---|---|---|---|
| | 0.000 | 2.118 | 4.868 | 11.668 | 20.868 |
| TR = 0.1 | 0.00% | 33.94% | 47.09% | 79.15% | 87.66% |
| TR = 0.3 | 0.00% | 28.84% | 41.99% | 74.05% | 82.56% |
| TR = 0.5 | 0.00% | 22.29% | 35.44% | 67.50% | 76.00% |
| TR = 0.8 | 0.00% | 9.72% | 22.88% | 54.94% | 63.44% |

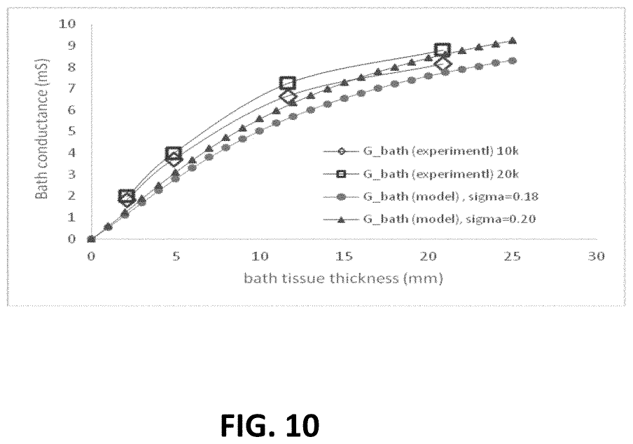

The experimental results of the parallel conductance (measured for two frequencies of 10 and 20 KHz) and the model predictions for the two bath conductivities (both similar to the conductivity of 0.1% saline solution used in the experiments) are plotted in FIG. 10. The agreement is very good for various surrounding tissue thicknesses.

Additional information regarding certain figures is as follows:

FIG. 5 shows 0.45% saline solution conductivity correction factor (BCCF) as a function of lumen diameter for two different guidewire diameters. BCCF is defined as the ratio of real to ideal conductivities of the blood.

FIG. 6 shows vessel wall tissue conductivity correction factor (TCCF) as a function of lumen diameter for 0.45% saline solution (TR=0.4). TCCF is defined as the ratio of real to ideal conductivities of the tissue.

FIG. 7 shows the ratio of parallel conductance to the conductance inside the lumen for an infinite tissue thickness as function of lumen diameter. The difference in the plots are due to the difference of conductivity inside the lumen.

FIG. 8 shows predicted lumen diameter versus optically measured for 11 in vitro tests with the line of equality.

FIG. 9 shows Bland-Altman chart for the 11 in vitro tests.

FIG. 10 shows a comparison of measured parallel conductance at 10 and 20 KHz, with model prediction as function of bath thickness.

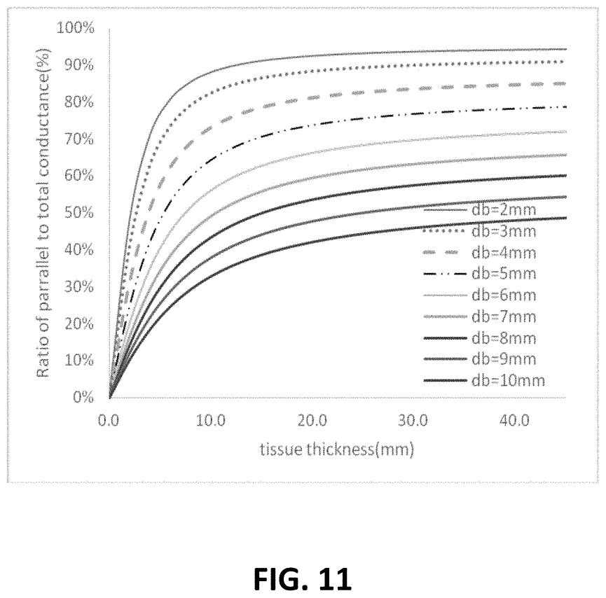

FIG. 11 shows the ratio of parallel conductance to the total conductance vs tissue thickness for various lumen diameters for 5-5-5 guidewire based on model prediction. There is 0.45% saline solution in the lumen and 0.1% saline solution in the tissue which closely represent the in vivo condition.

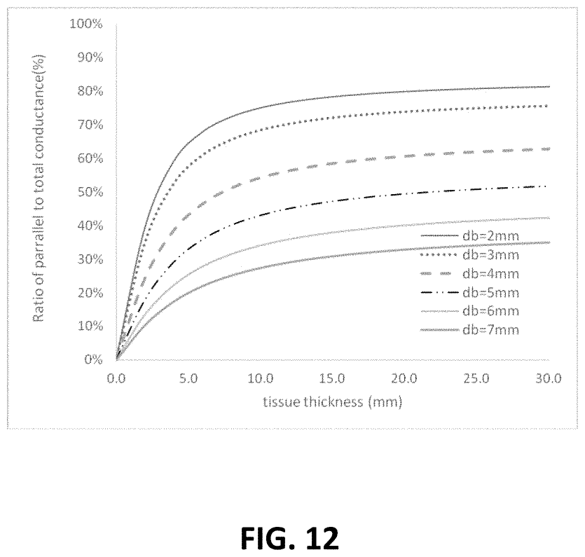

FIG. 12 shows the ratio of parallel conductance to the total conductance vs tissue thickness for various lumen diameters for 2-2-2 guidewire based on the model prediction. There is 0.45% saline solution in the lumen and 0.1% saline solution in the tissue which closely represent the in vivo condition.

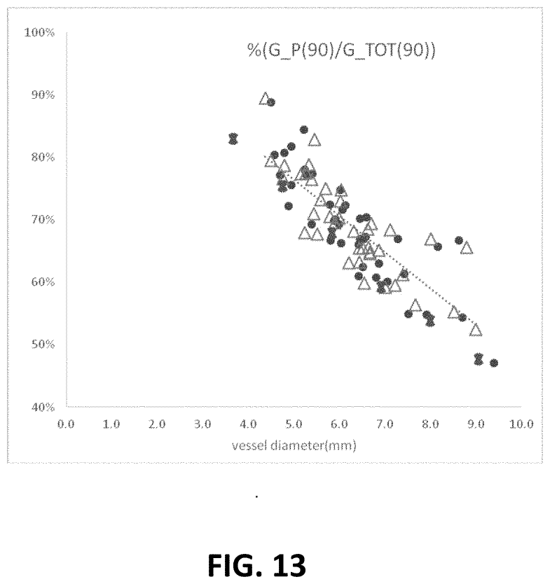

FIG. 13 shows the ratio of parallel conductance to total conductance as function of vessel diameter for 25 patients with the two-injections method using 5-5-5 guidewire. The points shown by cross marks are the values predicted by the model.

### DISCUSSION

The two-frequency approach was applied to calculate 0.45% saline solution conductivities at various frequencies for various phantom diameters. The conductivity increases with frequency and decreases with diameter. The model was also used to calculate the vessel wall tissue conductivity. The tissue conductivity is relatively constant in the frequency range of interest.

The two-frequency approach was used to study the relative contribution of parallel conductance for various diameters and tissue thicknesses. The vessel wall conductance was accounted for as part of the parallel conductance. FIG. 11 shows the ratio of the parallel conductance to the total conductance as function of tissue thickness for various diameters. The system consists of 0.45% saline solution in the lumen and 0.1% saline solution outside of the lumen which represents the surrounding tissue. This set up mimics the in vivo conditions. The 0.45% solution conductivities were obtained in phantom tests as reported here. The bath conductivity is assumed to be 0.18 S/m for these figures which corresponds to 0.1% saline solution conductivity at room temperature. Most of the electrical conductance flows outside of the lumen at lower lumen diameters which makes the contribution of the lumen conductance to the total conductance negligible for the 5-5-5 guidewire. For the smaller vessels, the parallel conductance is reduced with the 2-2-2 guidewire as shown in FIG. 12 where the excitation and the sensing lengths are smaller. The figure demonstrates the advantage of using 2-2-2 guidewire for the smaller vessels due to the lower ratio of the parallel conductance to the total conductance while the 5-5-5 guidewire is more appropriate for the larger vessels since they have lower parallel conductance and the electrical field can maintain cylindricity in the larger vessels.

One of the assumptions of the two-injections method is that the parallel conductance is the same when injecting two saline solutions at different electrical conductivities. The electrical conductivities of the two saline solutions differ by a factor of two. The parallel conductance model developed here can be used to validate the two-injections method assumption. This exercise has been applied to the clinical data taken for 25 patients at different anatomical locations with the two-injections method and 5-5-5 guidewire. The vessel diameters vary between 4 and 9 mm. The ratio of parallel conductance to the total conductance as function of vessel diameter is shown in FIG. 13 for the case of 0.9% saline solution. The total conductance in this figure has been measured and there are two measurements at each location. The parallel conductance has been derived by subtracting the total conductance from the vessel conductance. The vessel conductance can be derived by the vessel diameter estimated from the two saline solution injections. It can be noted that this ratio drops with increase in diameters. An estimate for the tissue conductivity of the patients' vessel is needed to be able to compare the data with the model since this is an input to the model. The tissue conductivity value was estimated by equating the ratio of the parallel conductance to the total conductance in FIG. 11 with the average ratio obtained in FIG. 13 for only one diameter. A vessel diameter of 5 mm was chosen for this purpose. It could be any diameter. The points shown by cross marks in FIG. 13 is the model predictions. The results show that the assumption of equal parallel conductance can be justified as predicted by the model.

Since the surrounding tissue conductivity and the width of the surrounding tissue can only be estimated in vivo, a sensitivity analysis can be performed to relate the changes prediction of lumen diameter. For surrounding tissue conductivity changes of 0.05 S/m from 0.20 S/m to 0.15 S/m (25% change), the ratio of parallel conductance to the total conductance changes only by around 4% for 2-2-2 guidewire. This translates into 2% change in lumen diameter since the lumen diameter is proportional to the square root of the lumen conductance. This amount of change is the same for different lumen diameters and is independent of the surrounding tissue thickness. This reduction of surrounding tissue conductivity can represent the existence of a fat tissue layer outside of the coronary wall tissue where the conductivity of fata is around 0.07 S/m which would reduce the overall conductivity of the surrounding tissue. For surrounding tissue thickness changes from 20 mm to 10 mm, the ratio changes by 0.04-0.06% depending on the lumen diameter (smaller changes for small diameters).

In brief summary, the present disclosure includes disclosure of a method that was devised to estimate the lumen diameter of the arteries without saline injections. In vitro data analysis demonstrates that this technique is a viable method to replace the injection method. The tool can also allow real-time pullback profiles of vessel lumen with unprecedented accuracy to guide therapy delivery for treatment of vascular disease.

While various embodiments of devices and methods of using the same have been described in considerable detail herein, the embodiments are merely offered as non-limiting examples of the disclosure described herein. It will therefore be understood that various changes and modifications may be made, and equivalents may be substituted for elements thereof, without departing from the scope of the present disclosure. The present disclosure is not intended to be exhaustive or limiting with respect to the content thereof.

Further, in describing representative embodiments, the present disclosure may have presented a method and/or a process as a particular sequence of steps. However, to the extent that the method or process does not rely on the particular order of steps set forth therein, the method or process should not be limited to the particular sequence of steps described, as other sequences of steps may be possible. Therefore, the particular order of the steps disclosed herein should not be construed as limitations of the present disclosure. In addition, disclosure directed to a method and/or process should not be limited to the performance of their steps in the order written. Such sequences may be varied and still remain within the scope of the present disclosure.

1. A method, comprising the steps of:
introducing at least part of an impedance device into a luminal organ at a first location so that a detector of the device is positioned within the luminal organ;
introducing a first frequency through the detector of the device and obtaining a first conductance measurement using the detector in connection with the first frequency;
introducing a second frequency through the detector of the device and obtaining a second conductance measurement using the detector in connection with the second frequency; and
determining a diameteror cross-sectional area at the first location within the luminal organ using the first conductance measurement, the second conductance measurement, the conductivity of fluid within the luminal organ, and a known distance between detection elements of the detector, and without injecting a fluid into the luminal organ in order to obtain the first conductance measurement and the second conductance measurement.

2. The method of claim 1, further comprising the step of:
generating a size profile of the luminal organ using the determined diameter or cross-sectional area at the first location and at least one additional diameter or cross-sectional area obtained by performing the steps of the method at a second location within the luminal organ.

3. The method of claim 1, wherein the conductivity of fluid within the luminal organ is determined by operating the detector of the device within a catheter positioned within the luminal organ by obtaining a conductance measurement within the catheter having a known diameter.

4. The method of claim 1, wherein the step of introducing at least part of the impedance device is performed to position the at least part of the device into the luminal organ wherein the detector comprises the two detection electrodes positioned in between two excitation electrodes, wherein the known distance between each of the electrodes is 5 mm.

5. The method of claim 1, wherein the steps of introducing the first frequency and introducing the second frequency are performed by operating a frequency generator in communication with the device, the frequency generator selected from the group consisting of an arbitrary waveform generator and multiple signal generators.

6. The method of claim 1, wherein the determining step is further performed to determine a parallel tissue conductance.

7. The method of claim 1, wherein the step of introducing at least part of the impedance device is performed to position the at least part of the device into the luminal organ wherein the detector comprises the two detection electrodes positioned in between two excitation electrodes, wherein the known distance between each of the electrodes is 2 mm.

8. The method of claim 1, wherein the first frequency and the second frequency are the only frequencies introduced.

9. The method of claim 1, further comprising the steps of:
moving the at least part of the impedance device within the luminal organ to a second location;
introducing one of the first frequency or the second frequency through the detector of the device and obtaining a third conductance measurement using the detector in connection with the one of the first frequency or the second frequency;
introducing the other of the first frequency or the second frequency through the detector of the device and obtaining a fourth conductance measurement using the detector in connection with the other of the first frequency or the second frequency;
determining a second diameter or cross-sectional area at the second location within the luminal organ using the third conductance measurement, the fourth conductance measurement, the conductivity of fluid within the luminal organ, and the known distance between detection elements of the detector, and without injecting a fluid into the luminal organ in order to obtain the third conductance measurement and the fourth conductance measurement; and
generating a size profile of the luminal organ using the determined diameter or cross-sectional area at the first location and at the determined second diameter or cross-sectional area at the second location.

10. A method, comprising the steps of:
introducing at least part of an impedance device into a luminal organ so that a detector of the device is positioned within the luminal organ;
operating the detector of the device within a catheter having a known diameter to obtain a first conductance measurement, wherein the catheter is positioned within the luminal organ and filled with a fluid native to the luminal organ;
operating the detector of the device to obtain a second conductance measurement, wherein the detector is positioned within the luminal organ and out of the catheter;
determining an initial estimate of the diameter or cross-sectional area of the luminal organ; and
determining an actual diameter or cross-sectional area of the luminal organ based on the first conductance measurement, the second conductance measurement, and the initial estimate of the diameter or cross-sectional area of the luminal organ.

11. The method of claim 10, wherein the steps of operating the detector of the device comprises introducing a first frequency through the detector of the device and introducing a second frequency through the detector of the device.

12. The method of claim 11, wherein the first frequency and the second frequency are the only frequencies introduced.

13. The method of claim 12, performed without injecting any fluid into the luminal organ.

14. The method of claim 14, further comprising the step of calculating at least one parallel conductance measurement based on the initial estimate of the diameter or cross-sectional area of the luminal organ.

15. The method of claim 14 further comprising the step of calculating a second parallel conductance measurement based on the initial estimate of the diameter or cross-sectional area of the luminal organ.

16. A method, comprising the steps of:
operating an impedance device to introduce a signal through the detection device into a luminal organ, the signal comprising a first signal having a first frequency, a second signal having a second frequency, and obtaining output conductance data in connection with each of the two signals using an impedance detector of the impedance device; and
determining a diameteror cross-sectional area of the luminal organ based upon the output conductance data in connection with each of the two signals and a conductivity of blood within the luminal organ;
wherein the method is performed without injecting any fluid into the luminal organ; and
wherein the signals are comprised of only the first signal and the second signal.

17. The method of claim 16, wherein the step of operating the impedance device to introduce a signal is performed repeatedly while the device is in motion.

18. The method of claim 16, wherein the step of operating the impedance device is performed to operate the impedance detector that comprises the two detection electrodes positioned in between two excitation electrodes, wherein the known distance between each of the electrodes is selected from the group consisting of 2 mm and 5 mm.

19. The method of claim 1, further comprising the steps of:
moving the impedance device within the luminal organ to a second location;
operating an impedance device to introduce the signal through the detection device into a luminal organ, the signal comprising the first signal having the first frequency and the second signal having the second frequency, and obtaining additional output conductance data in connection with each of the two signals using the impedance detector of the impedance device; and
determining a second diameter or cross-sectional area of the luminal organ based upon the additional output conductance data in connection with each of the two signals and the conductivity of blood within the luminal organ.

20. The method of claim 16, wherein the step of determining the diameter or cross-sectional area of the luminal organ is performed based upon an initial estimate of the luminal organ diameter or cross-sectional area.
